(19) United States
(12) Patent Application Publication
(10) Pub. No.: US 2015/0296758 A1
(43) Pub. Date: Oct. 22, 2015

(54) HUMANIZED TRANSGENIC SINGLE NUCLEOTIDE POLYMORPHISM ANIMAL SYSTEMS

(71) Applicant: THE JOHNS HOPKINS UNIVERSITY, Baltimore, MD (US)

(72) Inventor: Jay Bream, Timonium, MD (US)

(21) Appl. No.: 14/648,011

(22) PCT Filed: Dec. 3, 2013
(86) PCT No.: PCT/US2013/072776

§ 371 Date: May 28, 2015

Related U.S. Application Data
(60) Provisional application No. 61/732,557, filed on Dec. 3, 2012.

(51) Int. Cl. A01K 67/027; C12N 9/02; C07K 14/715; C07K 14/54; C07K 14/705; C12N 9/04; C12N 9/80

(52) U.S. Cl. CPC A01K 67/0278; C12N 9/0006; C12N 9/0073; C12N 9/80; C07K 14/5428; C07K 14/54; C07K 14/70596; C07K 14/7151; A01K 2267/0368; A01K 2227/105; A01K 2227/101; A01K 2227/103; A01K 2227/30; A01K 2227/108; A01K 2227/40; A01K 2217/056; C12Y 101/04001; C12Y 305/01098

(57) ABSTRACT

The present invention relates to the field of transgenic animals. More specifically, the present invention provides methods and composition related to humanized transgenic single polymorphism non-human animal systems. In one embodiment, a system comprises (a) a transgenic non-human animal comprising a transgene encoding a wildtype human protein, wherein the protein is biologically active in the animal; and (b) at least one transgenic non-human animal comprising a transgene encoding a variant human protein, wherein the protein is biologically active in the animal and wherein the variant comprises one or more single nucleotide polymorphisms (SNPs).

### CROSS-REFERENCE TO RELATED APPLICATIONS

This application claims the benefit of U.S. Provisional Application No. 61/732,557, filed Dec. 3, 2012, which is incorporated herein by reference in its entirety.

### STATEMENT OF GOVERNMENTAL INTEREST

This invention was made with U.S. government support under grant no. R01 AI070594. The U.S. government has certain rights in the invention.

### FIELD OF THE INVENTION

The present invention relates to the field of transgenic animals. More specifically, the present invention provides methods and composition related to humanized transgenic single polymorphism mouse systems.

### INCORPORATION-BY-REFERENCE OF MATERIAL SUBMITTED ELECTRONICALLY

This application contains a sequence listing. It has been submitted electronically via EFS-Web as an ASCII text file entitled “P12089-02_ST25.txt.” The sequence listing is 711,783 bytes in size, and was created on Dec. 3, 2013. It is hereby incorporated by reference in its entirety.

### BACKGROUND OF THE INVENTION

In microorganisms, selective pressure, provided by antibiotics for example, results in the emergence of new strains that have been selected for based on natural genetic mutations that provide a survival advantage (i.e., drug-resistant strains). The same is true in higher organisms exposed to infectious microorganisms, and the net result of these natural evolutionary processes has been the development of host genetic diversity that is evidenced by differential disease susceptibility in human populations. Human genetic variation does not only impact disease risk, but also has a profound influence on vaccine efficacy and how individuals respond to medication. Collectively, this has spurred enormous growth in the relatively new field of pharmacogenomics, which is defined as the study of how genetic variability in human populations impacts on an individual's response to medicines. Often, this can be traced to the expression of genes in target pathways. In recent years, many groups in academia and industry have made efforts to exploit the in vivo capabilities of mouse models to understand how human genes are regulated by utilizing transgenic approaches to introduce human DNA into mice. Although this method can offer valuable insight into the basic molecular mechanisms that govern gene expression, it has some limitations. First, transgenic systems do not account for the influence of genetic variation on gene regulation at a given loci. Secondly, reporter genes are generally used to monitor gene activity and are therefore not biologically active. These factors limit the ability to study the downstream biological effects of gene induction on the intact animal.

Thus, there are very few current models that can address both of these important issues which would be of great value to academia, biotech, and the pharmaceutical industry as such models would permit the in vivo evaluation of human gene expression and function in response to infectious microorganisms, vaccines, and experimental therapies.

### SUMMARY OF THE INVENTION

The present invention is based, at least in part, on the development of a transgenic non-human animal system that permits the in vivo evaluation of human gene expression and function in response to infectious microorganisms, vaccine and experimental therapies. The present invention addresses the limitations of current approaches described above and provides great value to academia, as well as the biotech and pharmaceutical industries.

The transgenic non-human animal system of the present invention was developed using the human IL-10 gene. Because IL-10 is linked to numerous autoimmune and infectious diseases, and cancers, and there are well-characterized IL-10-dependent models of such diseases (i.e., colitis, toxoplasmosis, leishmaniasis, sepsis, multiple sclerosis, etc.), the transgenic system of the present invention offers an ideal in vivo model for pre-clinical testing of drug candidates targeting (human) IL-10 in general but can also be used to investigate pharmacogenomic dynamics of therapeutics based on different human IL10 alleles. Indeed, the present invention permits the evaluation of pharmacogenomic influence of therapeutics or vaccines on human gene expression and influence on disease outcomes in intact animals ideally for a clear in vivo model system for pre-clinical evaluation of therapeutics which target (human) IL-10.

Although the instant application is described in the context of IL-10, the present invention is not so limited. Other DNA modifications are possible to include other known DNA haplotypes in this locus. More generally, this technique could be applied to include other BACs which would include another human gene of interest, all genomic regulatory regions, and a functional gene product which would be the basis for future BAC lines generated of the same length but with a different genetic haplotype. In a broader sense, this approach represents an alternative to current attempts to generate “humanized” mice. Indeed, the present invention represents a powerful research tool for academia and industry, particularly given the ability to evaluate the role of different human genetic haplotypes on gene expression and disease outcomes in vivo.

Accordingly, in one aspect, the present invention provides systems of transgenic non-human animals. In one embodiment, a system comprises (a) a transgenic non-human animal comprising a transgene encoding a wildtype human protein, wherein the protein is biologically active in the animal; and (b) at least one transgenic non-human animal comprising a transgene encoding a variant human protein, wherein the protein is biologically active in the animal and wherein the variant comprises one or more single nucleotide polymorphisms (SNPs). In alternative embodiments, the variant human protein can comprise an insertion or a deletion. Indeed, it is contemplated that the variant human protein comprises a mutation relative to the wildtype or reference version. In other embodiments, the variant human protein can comprise a combination of SNPs, insertions and/or deletions. In particular embodiments, the transgenic non-human animal is murine, bovine, ovine, porcine, avian or piscine. In other specific embodiments, the human protein is a cytokine, cytokine receptor, TNF receptor, drug metabolizing enzyme, inflammatory protein or chromatin/DNA modifying protein. The term “wildtype” is used interchangeably with the term “reference.” For example, hIL10BAC-GCC (modified BAC) is compared to a reference genome for identification of variants. An example of a reference genome for IL-10 is shown in SEQ ID NO:3.

In certain embodiments, the transgene is a bacterial artificial chromosome (BAC) comprising a nucleotide sequence encoding the wildtype or variant human protein. In other embodiments, the transgene encoding the wildtype or variant human protein comprises the non-coding regulatory regions of the human protein. In further embodiments, the one or more SNPs are located in the promoter of the nucleotide sequence encoding the variant human protein.

In another embodiment, a system comprises (a) a transgenic non-human animal comprising a transgene encoding a wildtype human protein, wherein the protein is biologically active in the animal; and (b) at least one transgenic non-human animal comprising a transgene encoding a variant human protein, wherein the protein is biologically active in the animal and wherein the variant comprises one or more mutations. In certain embodiments, the one or more mutations can comprise one or more of SNPs, insertions, and/or deletions.

In a specific embodiment, a system comprises (a) a transgenic mouse comprising a transgene encoding a wildtype human protein, wherein the protein is biologically active in the animal; and (b) at least one transgenic mouse comprising a transgene encoding a variant human protein, wherein the protein is biologically active in the mouse and wherein the variant comprises one or more SNPs. The human protein can be a cytokine, cytokine receptor, TNF receptor, drug metabolizing enzyme, inflammatory gene or chromatin/DNA modifying gene. In a specific embodiment, the human protein is interleukin-10 (IL-10). In another specific embodiment, the transgene is a bacterial artificial chromosome (BAC) comprising a nucleotide sequence encoding the wildtype or variant human protein. In yet another embodiment, the transgene encoding the wildtype or variant human protein comprises the non-coding regulatory regions of the human protein. In certain embodiments, the one or more SNPs are located in the promoter of the nucleotide sequence encoding the variant human protein.

In another aspect, the present invention provides transgenic non-human animals. In one embodiment, a transgenic non-human animal comprises a transgene encoding a variant human interleukin-10 (IL-10), wherein the variant comprises one or more single nucleotide polymorphisms (SNPs). In particular embodiments, the animal is murine, bovine, ovine, porcine, avian or piscine. In certain embodiments, the transgene is a bacterial artificial chromosome (BAC) comprising a nucleotide sequence encoding a variant human IL-10, wherein the variant comprises one or more SNPs. In other embodiments, the one or more SNPs are located in the IL-10 promoter. In a specific embodiment, the SNP is one or more of rs12569132, rs11809840, rs11809303, rs11808255, rs11802425, rs11802412, rs11799787, rs11585685, rs11579735, rs11119451, rs11119449, rs10494878, rs7556267, rs7548373, rs7537619, rs7519318, rs7512090, rs7418268, rs6692511, rs6682675, rs6673928, rs6668464, rs6668374, rs6540587, rs6540586, rs6540582, rs4844553, rs4844552, rs4579758, rs4483409, and rs11807715. In alternative embodiments, the one or more SNPs are located in the IL-10 gene. In more specific embodiments, the SNP is one or more of rs3024496, rs3024491, rs3024490, rs3021094, rs2222202, rs1800896, rs1800894, rs1800893, rs1800891, rs1800872, rs1800871, rs1554286, rs1518111, and rs1518110.

In a further embodiment, BAC further comprises a nucleotide sequence encoding mitogen activated protein kinase-activated protein kinase 2 (MK2). In a specific embodiment, the nucleotide sequence encoding MK2 comprises one or more SNPs located in the MK2 promoter. In particular embodiments, the SNP is one or more of rs11119297, rs7410826, rs4240843, and rs4240842. In an alternative embodiment, the nucleotide sequence encoding MK2 comprises one or more SNPs located within the MK2 gene. In exemplary embodiments, the SNP is one or more of rs12564851, rs12404831, rs12060808, rs11809485, rs11582798, rs11119390, rs11119389, rs11119385, rs11119355, rs10863805, rs10863788, rs10863787, rs10863784, rs7530164, rs6669284, rs6540574, rs6540548, rs4845132, rs4845131, rs4539133, rs4325131, rs4274064, rs4256810, rs4240848, rs4240847, and rs4072677.

Furthermore, in certain embodiments, the BAC further comprises a nucleotide sequence encoding IL-19. In a specific embodiment, the nucleotide sequence encoding IL-19 comprises one or more SNPs located in the IL-19 promoter. In certain embodiments, the SNP is one or more of rs12565617, rs12563100, rs11805284, rs11805136, rs11583398, rs11583394, rs11583394, rs11119570, rs10494879, rs6702254, rs6667202, rs4845141, rs4845140, rs4845138, rs4845136, rs4845135, rs4845134, rs4072227, rs4072226, rs3122605, rs3001101, rs2945417, rs2015273, rs885334, rs11581469, rs11119515, and rs11119514.

In another embodiment, the nucleotide sequence encoding IL-19 comprises one or more SNPs within the IL-19 gene. In particular embodiments, the SNP is one or more of rs12409785, rs12409577, rs12409415, rs12408017, rs12407485, rs12407461, rs12040948, rs11811600, rs11811158, rs11802960, rs11799303, rs11119629, rs11119623, rs11119622, rs11119621, rs11119619, rs11119587, rs11119585, rs11119584, rs11119582, rs10863863, rs10863859, rs10746433, rs7529836, rs7518426, rs7513988, rs6685379, rs6663563, rs6660537, rs6660520, rs6540645, rs4845143, rs4440852, rs4347211, rs4240849, rs3950619, rs2883035, rs2352794, rs2243183, rs2243170, rs2243168, rs2243158, rs2243156, rs2056225, rs1878673, and rs1028182.

In a further embodiment, the BAC further comprises a nucleotide sequence encoding IL-20. In another embodiment, the nucleotide sequence encoding IL-20 comprises one or more SNPs located in the IL-20 promoter. In certain embodiments, the SNP is one or more of rs908704, rs908703, rs1798, rs1028182, rs7532642, rs7530109, rs11809303, rs11807894, rs11119726, rs10863890, rs10863889, rs7532642, rs3860299, rs1713233, rs1033272, rs570249, rs552760, and rs523435.

In another aspect, the present invention provides methods for screening agents. In a specific embodiment, a method for screening a candidate agent for the ability modulate IL-10 expression in the transgenic non-human animal of the present invention comprises the steps of (a) administering to a first transgenic non-human animal a candidate agent; and (b) comparing IL-10 expression in the first transgenic non-human animal to IL-10 expression of a second transgenic non-human animal of not administered the candidate agent, wherein a difference in the IL-10 expression in the first transgenic non-human animal administered the candidate agent compared to the second transgenic non-human animal not administered the candidate agent is indicative of a candidate agent that modulates IL-10 expression. In a specific embodiment, the transgenic non-human animal further comprises a disease model that is associated with IL-10. In another embodiment, a difference in disease severity is also compared. In other embodiments, the steps can be repeated using transgenic non-human animals that comprise a wildtype or reference human protein. The method can comprise repeating the steps with different transgenic non-human animals that express, for example, wildtype/reference protein and variants thereof.

Again, this example is not so limited to IL-10, as one skilled in the art can construct transgenic non-animal systems that express other genes that encode human proteins including, but not limited, cytokine genes, cytokine receptor gene(s), TNF receptor genes, drug metabolizing enzyme genes, inflammatory genes, HLA genes, cancer genes/oncogenes, autophagy genes, cell death genes, and chromatin/DNA modifying genes.

In another specific embodiment, the present invention provides a system comprising (a) hIL10BAC-ATA mice and (b) hIL10BAC-GCC mice. In a specific embodiment, the hIL10BAC-ATA mice comprise a BAC encoded by SEQ ID NO:1. In another specific embodiment, the hIL10BAC-GCC mice comprise a BAC encoded by SEQ ID NO:2. In a further embodiment, the system further comprises mice that comprise a transgene encoding a BAC comprising human wild type IL10 gene and regulatory regions. In yet another embodiment, a transgenic non-human animal comprises a transgene encoding a human protein known to influence disease susceptibility, wherein the protein is biologically active in the animal.

### BRIEF DESCRIPTION OF THE FIGURES

FIG. 1: Initial PCR analysis of BACs CTD-2563L3 and CTD-3174K1. Before carrying the DNA transplantation into execution PCR checks across the intended modification junctions were made. A) Schematic representation of the “donor” BAC CTD-2563L3 and the “acceptor” BAC CTD-3174K1. The position of three check PCR reactions on the BACs are given as red marks on the outer circles. Check primer pairs 1+2 and 4+5 amplify 5′ and 3′ flanking region of the “missing portion” (pink box). B) Analysis of PCR reactions by agarose gel electrophoresis. M: DNA size marker.

FIG. 2: Replacement of 30 kb downstream of the “missing portion”. A) Schematic representation of Red/ET mediated homologous recombination to replace a 30 kb segment of the donor BAC CTD-2563L3 by a 985 bp KanR cassette. 50 bp homology regions are given in red (cassette not to scale). B) Analysis of the replacement cassette by agarose gel electrophoresis. Using the primer pairs P344-PCR1-up×P344-PCR1-down (lane 1) on an appropriate gb2-KanR template the replacement cassette of 985 bp was generated. Samples were analyzed on a 1% (w/v) agarose gel. M: DNA size marker

FIG. 3: Flank check PCR after replacement of a 30 kb BAC section by a KanR cassette in CTD-2563L3. A) Map of CTD-2563L3-KanR. B) Schematic overview on the applied PCR strategy. C) Agarose gel electrophoresis of flank check PCRs on five clones derived from the recombination described in chapter 3. Using the primer pairs P344-check4×GBPR101 (5′ lanes) and GBPR75×GBPR61 (3′ lanes) on twelve putative recombinants (only five shown) integration of the KanR cassette was confirmed in all cases. Samples were analyzed on a 1% (w/v) agarose gel. M: DNA size marker.

FIG. 4: Subcloning of the “missing portion” into a low copy vector. A) Schematic representation of Red/ET mediated homologous recombination for subcloning of a 15.6 kb segment (“missing portion” plus KanR gene) from the truncated donor BAC CTD-2563L3-KanR into a linear minimal vector. 50 bp homology regions are given in red (not to scale). B) Analysis of the linear minimal vector by agarose gel electrophoresis. Using the primer pairs P344-PCR2-up×P344-PCR2-down (lane 1) on pACYC177 the minimal vector of 2141 bp was generated. Samples were analyzed on a 1% (w/v) agarose gel. M: DNA size marker.

FIG. 5: Restriction analysis of subclone “missing portion”. A) Map of the subclone. The “missing portion” of 12.6 kb is given in pink. Other elements: Kanamycin resistance gene (KanR): yellow; Ampicillin resistance gene (AmpR): red; p15A origin of replication: gold. Position of recognition sites of the three restriction enzymes PmeI, SacI, and BglII are indicated. B) Representative analysis of restriction digestion of the sucblone by agarose gel electrophoresis. Expected fragment sizes: lane 1-PmeI: 15.6 kb, 2049 bp; lane 2-SacI: 5481 bp, 4353 bp, 3555 bp, 1888 bp, 1601 bp, 642 bp, 183 bp (the last two fragment are not visible in the gel); lane 3-BglII: 6727 bp, 6480 bp, 3354 bp, 1142 bp. Samples were analyzed on a 1% (w/v) agarose gel. M: DNA size marker.

FIG. 6: Flank check PCR after insertion of the “missing portion” into BAC CTD-3174K1. A) Map of CTD-3174K1+12.6 kb. B) Schematic overview on the applied PCR strategy. C) Agarose gel electrophoresis of flank check PCRs on one clones derived from the recombination descried in the text. Using the primer pairs GBPR75×GBPR97 (5′ lane) and P344-check1×P344-check2 (3′ lane) on six putative recombinants (only one shown) integration of the missing portion was confirmed in all cases. Samples were analyzed on a 1% (w/v) agarose gel. M: DNA size marker.

FIG. 7. Diagram of BAC clones used for hIL10BAC lines and strategy for recombineering 12.6 Kb insert into parent GCC BAC to make hIL10BAC-GCC construct. HR-homologous recombination.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

It is widely appreciated that excessive immune activation results in an increased risk of disease morbidity and mortality. The anti-inflammatory cytokine IL-10 is a key regulator of inflammation by limiting excessive host responses to pathogens. In humans, hundreds of studies have described strong associations between IL-10 and risk for a wide array of infectious and autoimmune diseases. These associations are based primarily on IL-10 levels and/or SNP haplotype blocks in the IL10 promoter. In fact, IL-10 levels are linked with promoter SNPs and human IL-10 production can be stratified from low (“IL10ATA”) to high (“IL10GCC”) based on these IL10 promoter haplotypes. The indication that IL-10 production is determined on a genetic basis is supported by the concordance of IL-10 levels in monozygotic twins, which suggests that between 50 and 70% of IL-10 production is genetically determined. This is likely a common evolutionary mechanism to promote diversity in human gene expression and in response to selective pressures which has been noted in multiple human cytokine loci including IL10. Furthermore, our group and others have found that transcription factors differentially bind IL10 promoter SNPs suggesting a mechanism for allele-specific IL-10 expression. Altogether, these data suggest that the basis for how IL-10 influences susceptibility to disease in humans is due, at least in part, to differences in how IL-10 expression is controlled.

While mouse models have been extremely useful in describing the biologic properties of IL-10 and the distributions of IL-10-expressing cell types in tissues, they cannot account for inter- and intra-species differences in IL-10 gene regulation. Indeed, divergence in gene regulation patterns between species has been attributed largely to difficulties in translating some experimental findings in mice to humans. A key to overcoming this obstacle is to develop new tools to study hIL-10 and human immunology in general.

The present inventors established a novel system to model hIL-10 regulation using a transgenic bacterial artificial chromosome-based approach (hIL10BAC). This strategy is based on the fact that while differences in regulatory DNA sequences drive species-specific gene expression, the transcriptional machinery itself is highly conserved. Accordingly, the present inventors have verified that tissue-specific gene expression can be transferred across species to mice. Thus, the mouse gene regulatory machinery is able to decode human regulatory DNA sequences which drive cell- and species-specific gene expression. In addition, the hIL-10 protein functionally binds the mouse IL-10R, thus enabling the assessment of hIL-10 gene regulation and function using mouse models of human disease. The hIL10BAC is the first model which permits the in vivo assessment of inter-individual variation in human gene expression and the ensuing consequences on disease outcomes.

In mouse models, a growing body of evidence indicates that different cellular sources of IL-10 play distinctive roles in disease pathology. For example, myeloid-derived IL-10 confers protection to LPS challenge and IL-10-producing Th1 cells control susceptibility to several intracellular parasites. The cellular sources of IL-10 are challenging to characterize in humans due to wide inter-individual variation in hIL-10 expression and technical limits in obtaining tissue samples resulting in a limited understanding of hIL-10 regulation in different cell types in relation to disease outcomes. Still, the molecular mechanisms regulating IL-10 production in both humans and mice are not understood. The present invention directly addresses the molecular and genetic basis of how cell- and allele-specific hIL-10 expression controls disease outcomes.

Altogether, the work described herein builds on these data and expands the approach to understand how human IL-10 gene regulation programs in different cell types contribute to disease outcomes. The present inventors hypothesize that hIL-10 expression is determined by cell- and allele-specific regulatory elements in the IL10 locus. The work described herein was designed to clarify the molecular and genetic mechanisms which control the temporal/spatial expression of hIL-10. By identifying the cellular sources, tissue locations, and genetic factors that impinge on hIL-10 production, it is possible to design more targeted, personalized therapeutic strategies to manage inflammation by manipulating hIL-10 expression.

Using the hIL10BAC transgenic system, the present inventors are dissecting the complexities of hIL-10 regulation in vivo and the subsequent impact on immunopathology. The hIL10BAC approach is highly multidisciplinary, integrating concepts and experimental tools from several disciplines including immunology, human genetics, gene regulation and disease pathology. The hIL10BAC model provides an exclusive opportunity to examine the relationship between the temporal/spatial regulation of hIL-10 production and disease outcomes.

In addition, this strategy provides a more generalizable foundation for modeling human gene expression and function in vivo. This methodology can be applied more broadly to other disciplines and include practical applications (i.e., pre-clinical screening of drugs targeting human proteins or pathways which induce human proteins, etc.). For over 25 years, scientists have attempted to “humanize” mice by various methods with little success. The present inventors have developed a robust tool to experimentally link human gene expression with function by using human chromosomal DNA constructs with functional genes (as opposed to reporter genes or human genes under the control of mouse chromosomal DNA) that offers unique insights into human health.

Accordingly, in certain embodiments, the present invention could be of great use for pre-clinical evaluation of drug efficacy in vivo and in the context of disease. Indeed, the mice described herein can be used to study the effect of human IL-10 in numerous various mouse models of inflammatory diseases. In further embodiments, a similar strategy can be used to generate other transgenic mice with functional human genes under genomic control as described herein. In more particular embodiments, human genes that are known or likely targets for therapeutic intervention (based on their biologic function) are of particular interest.

The human IL-10 gene was selected for several reasons. First, IL-10 is associated with various autoimmune, infectious diseases and cancers in humans. Diseases are associated with single nucleotide polymorphisms (SNPs) in the IL10 promoter as well as levels of IL-10 protein production. Second, human IL-10 production is determined largely on a genetic basis. IL-10 promoter SNP haplotypes are associated with IL-10 protein levels (i.e., high vs. low IL-10). Third, mouse IL-10 expression and function is well-characterized. Fourth IL-10 deficiency in mice is associated with clear phenotypes which are mediated by tissue-specific expression of IL-10. Finally, human IL-10 protein is biologically active in the mouse and is easily distinguished.

The present inventors have generated new transgenic lines to address the role of SNPs on IL-10 conferring differential disease risk to inflammatory diseases. The present invention places the expression of functional human genes under genomic control in the context of different well-defined genotypes that are known to influence disease susceptibility and possibly drug efficacy. A system is now in place to determine the role of genetic variation in disease outcomes as well as response to therapeutics (for which this gene is frequently targeted) in a critical anti-inflammatory gene.

Human IL10 SNPs Affect Cell-Specific hIL-10 Production and Disease Outcomes.

Variations (such as SNPs) in non-coding regions of DNA that regulate gene expression are thought to be a driving force in evolution and individual differences in disease outcomes. Non-coding SNPs can impact gene expression and phenotypic variation by altering chromatin structure. Importantly, these variations affect gene expression by altering the binding of transcription factors to specific DNA motifs that has been documented between and within species.

SNPs in the human IL10 promoter are associated disease outcomes. The majority of gene association studies focus on a series of 3 SNPs in the proximal IL10 promoter consisting of −1082G/A (rs1800896), −819C/T (rs1800871), and −592C/A (rs1800872). These SNPs are in tight linkage disequilibrium which results in well-defined haplotypes including IL10ATA and IL10GCC. The IL10ATA and IL10GCC haplotypes are also associated with low and high IL-10 production respectively. Thus, the genetic control of IL-10 levels is thought to mediate disease risk. After carefully validating the hIL10BAC model we are now well-positioned to address this concept in vivo utilizing a new generation of hIL10BAC mice.

The BAC comprises one or more IL-10 SNPs. Known or unknown SNPs can be used. Known SNPs can be accessed via one of a number of publicly available databases including, but not limited to, the NCBI HAPMAP database. In specific embodiments, the SNPs are within the genomic area from chromosome 1 that was used to generate the hIL10BAC mice. In particular embodiments, a human IL-10 transgenic mouse can comprise one or more SNPs located upstream or downstream of one or more of the three genes in the BAC described herein, specifically, mitogen activated protein kinase-activated protein kinase 2 (Mapkapk2 or MK2), interleukin 10 (IL10 or IL-10), and interleukin 19 (IL19 or IL-19).

In certain embodiments, a human IL-10 transgenic mouse can comprise one or more SNPs located 5′ of the MK2 gene including, but not limited to, rs11119297, rs7410826, rs4240843, and rs4240842.

In other embodiments, a human IL-10 transgenic mouse comprises one or more SNPs located within the MK2 gene including, but not limited to, rs12564851, rs12404831, rs12060808, rs11809485, rs11582798, rs11119390, rs11119389, rs11119385, rs11119355, rs10863805, rs10863788, rs10863787, rs10863784, rs7530164, rs6669284, rs6540574, rs6540548, rs4845132, rs4845131, rs4539133, rs4325131, rs4274064, rs4256810, rs4240848, rs4240847, and rs4072677.

In further embodiments, embodiments, a human IL-10 transgenic mouse comprises one or more SNPs located 5′ of the IL-10 gene (3′ of MK2) including, but not limited to, rs12569132, rs11809840, rs11809303, rs11808255, rs11802425, rs11802412, rs11799787, rs11585685, rs11579735, rs11119451, rs11119449, rs10494878, rs7556267, rs7548373, rs7537619, rs7519318, rs7512090, rs7418268, rs6692511, rs6682675, rs6673928, rs6668464, rs6668374, rs6540587, rs6540586, rs6540582, rs4844553, rs4844552, rs4579758, rs4483409, and rs11807715.

In yet other embodiments, a human IL-10 transgenic mouse comprises one or more SNPs located within the IL-10 gene including, but not limited to, rs3024496, rs3024491, rs3024490, rs3021094, rs2222202, rs1800896, rs1800894, rs1800893, rs1800891, rs1800872, rs1800871, rs1554286, rs1518111, and rs1518110.

In specific embodiments, a human IL-10 transgenic mouse comprises one or more SNPs located 5′ of the IL-19 gene (3′ of IL-10) including, but not limited to, rs12565617, rs12563100, rs11805284, rs11805136, rs11583398, rs11583394, rs11583394, rs11119570, rs10494879, rs6702254, rs6667202, rs4845141, rs4845140, rs4845138, rs4845136, rs4845135, rs4845134, rs4072227, rs4072226, rs3122605, rs3001101, rs2945417, rs2015273, rs885334, rs11581469, rs11119515, and rs11119514.

In particular embodiments, a human IL-10 transgenic mouse comprises one or more SNPs located within the IL-19 gene including, but not limited to, rs12409785, rs12409577, rs12409415, rs12408017, rs12407485, rs12407461, rs12040948, rs11811600, rs11811158, rs11802960, rs11799303, rs11119629, rs11119623, rs11119622, rs11119621, rs11119619, rs11119587, rs11119585, rs11119584, rs11119582, rs10863863, rs10863859, rs10746433, rs7529836, rs7518426, rs7513988, rs6685379, rs6663563, rs6660537, rs6660520, rs6540645, rs4845143, rs4440852, rs4347211, rs4240849, rs3950619, rs2883035, rs2352794, rs2243183, rs2243170, rs2243168, rs2243158, rs2243156, rs2056225, rs1878673, and rs1028182.

In other embodiments, a human IL-10 transgenic mouse comprises one or more SNPs located 5′ of the IL-20 gene (3′ of IL-19) including, but not limited to, rs908704, rs908703, rs1798, rs1028182, rs7532642, rs7530109, rs11809303, rs11807894, rs11119726, rs10863890, rs10863889, rs7532642, rs3860299, rs1713233, rs1033272, rs570249, rs552760, and rs523435.

In particular embodiments, a hIL10BAC mice can comprise one or more SNPs including, but not limited to, rs11119297, rs7410826, rs4240843, rs4240842, rs12564851, rs12404831, rs12060808, rs11809485, rs11582798, rs11119390, rs11119389, rs11119385, rs11119355, rs10863805, rs10863788, rs10863787, rs10863784, rs7530164, rs6669284, rs6540574, rs6540548, rs4845132, rs4845131, rs4539133, rs4325131, rs4274064, rs4256810, rs4240848, rs4240847, rs4072677, rs12569132, rs11809840, rs11809303, rs11808255, rs11802425, rs11802412, rs11799787, rs11585685, rs11579735, rs11119451, rs11119449, rs10494878, rs7556267, rs7548373, rs7537619, rs7519318, rs7512090, rs7418268, rs6692511, rs6682675, rs6673928, rs6668464, rs6668374, rs6540587, rs6540586, rs6540582, rs4844553, rs4844552, rs4579758, rs4483409, rs11807715, rs3024496, rs3024491, rs3024490, rs3021094, rs2222202, rs1800896, rs1800894, rs1800893, rs1800891, rs1800872, rs1800871, rs1554286, rs1518111, rs1518110, rs12565617, rs12563100, rs11805284, rs11805136, rs11583398, rs11583394, rs11583394, rs11119570, rs10494879, rs6702254, rs6667202, rs4845141, rs4845140, rs4845138, rs4845136, rs4845135, rs4845134, rs4072227, rs4072226, rs3122605, rs3001101, rs2945417, rs2015273, rs885334, rs11581469, rs11119515, rs11119514, rs12409785, rs12409577, rs12409415, rs12408017, rs12407485, rs12407461, rs12040948, rs11811600, rs11811158, rs11802960, rs11799303, rs11119629, rs11119623, rs11119622, rs11119621, rs11119619, rs11119587, rs11119585, rs11119584, rs11119582, rs10863863, rs10863859, rs10746433, rs7529836, rs7518426, rs7513988, rs6685379, rs6663563, rs6660537, rs6660520, rs6540645, rs4845143, rs4440852, rs4347211, rs4240849, rs3950619, rs2883035, rs2352794, rs2243183, rs2243170, rs2243168, rs2243158, rs2243156, rs2056225, rs1878673, rs1028182, rs908704, rs908703, rs1798, rs1028182, rs7532642, rs7530109, rs11809303, rs11807894, rs11119726, rs10863890, rs10863889, rs7532642, rs3860299, rs1713233, rs1033272, rs570249, rs552760, and rs523435.

In particular embodiments, a hIL10BAC mice can comprise one or more SNPs selected from the group consisting of rs11119297, rs7410826, rs4240843, rs4240842, rs12564851, rs12404831, rs12060808, rs11809485, rs11582798, rs11119390, rs11119389, rs11119385, rs11119355, rs10863805, rs10863788, rs10863787, rs10863784, rs7530164, rs6669284, rs6540574, rs6540548, rs4845132, rs4845131, rs4539133, rs4325131, rs4274064, rs4256810, rs4240848, rs4240847, rs4072677, rs12569132, rs11809840, rs11809303, rs11808255, rs11802425, rs11802412, rs11799787, rs11585685, rs11579735, rs11119451, rs11119449, rs10494878, rs7556267, rs7548373, rs7537619, rs7519318, rs7512090, rs7418268, rs6692511, rs6682675, rs6673928, rs6668464, rs6668374, rs6540587, rs6540586, rs6540582, rs4844553, rs4844552, rs4579758, rs4483409, rs11807715, rs3024496, rs3024491, rs3024490, rs3021094, rs2222202, rs1800896, rs1800894, rs1800893, rs1800891, rs1800872, rs1800871, rs1554286, rs1518111, rs1518110, rs12565617, rs12563100, rs11805284, rs11805136, rs11583398, rs11583394, rs11583394, rs11119570, rs10494879, rs6702254, rs6667202, rs4845141, rs4845140, rs4845138, rs4845136, rs4845135, rs4845134, rs4072227, rs4072226, rs3122605, rs3001101, rs2945417, rs2015273, rs885334, rs11581469, rs11119515, rs11119514, rs12409785, rs12409577, rs12409415, rs12408017, rs12407485, rs12407461, rs12040948, rs11811600, rs11811158, rs11802960, rs11799303, rs11119629, rs11119623, rs11119622, rs11119621, rs11119619, rs11119587, rs11119585, rs11119584, rs11119582, rs10863863, rs10863859, rs10746433, rs7529836, rs7518426, rs7513988, rs6685379, rs6663563, rs6660537, rs6660520, rs6540645, rs4845143, rs4440852, rs4347211, rs4240849, rs3950619, rs2883035, rs2352794, rs2243183, rs2243170, rs2243168, rs2243158, rs2243156, rs2056225, rs1878673, rs1028182, rs908704, rs908703, rs1798, rs1028182, rs7532642, rs7530109, rs11809303, rs11807894, rs11119726, rs10863890, rs10863889, rs7532642, rs3860299, rs1713233, rs1033272, rs570249, rs552760, and rs523435.

### Transgenic Animal Systems Comprising Other Genes.

The methods and compositions of the present invention can be used to express virtually any other gene or genes of interest. For example, the present invention also includes transgenic animal systems that comprise one or more genes including, but not limited to cytokine genes, cytokine receptor gene(s), TNF receptor genes, drug metabolizing enzyme genes, inflammatory genes, HLA genes, cancer genes/oncogenes, autophagy genes, cell death genes, and chromatin/DNA modifying genes.

In certain embodiments, transgenic animal systems of the present invention can comprise one or more genes encoding cytokines including, without limitation, IL1B, IL2, IL4, IL5, IL6, IL7, IL8, IL9, IL11, IL-2A, IL12B, IL13, IL15, IL17A, IL17B, IL17C, IL17E, IL18, IL19, IL20, IL21, IL22, IL23, IL24, IL27 (EBI3), IL27-p28, IL28A, IL28B, IL29, IL31, IL32, IL33, IFNB, IFNG, TNFA, TGFB, LT, LIF, EPO, OSM, GCSF, GMCSF, PRO, TSLP, and EGF.

Transgenic animal systems of the present invention can also comprise one or more genes encoding cytokine receptors including, but not limited to, IL1R, IL1R2, IL1RAPL2, IL1RA, IL2R, IL3R, IL4R, IL6R, IL7R, IL8R, IL9R, IL10RA/B, IL11R, IL12R, Il13R, IL15R, IL17R (subunits A, B, C, D, E encoding for IL17R), IL18R, IL20R, IL21R, IL22R, IL22BP, IL23R, IL27R, IL28R, TGFBR 1-3, IFNAR1/2, IFNGR1/2, GP130, LIFR (CD118), OSMR, EPOR, GMCSFR (CD116), IL31R, EGFR, PR, and TSLPR. In certain embodiments, receptors (cytokine, TNF, etc.) are usually comprised of more than one chain (i.e., more than one gene encodes for a functional receptor). In such cases, the genes encoding the subunits can be introduced into one or more vectors. For example, the genes encoding the subunits of a receptor can be cloned into one BAC or as many BACs as there are subunit-encoding genes.

In further embodiments, transgenic animal systems of the present invention can comprise one or more genes encoding TNF receptors including, but not limited to, TNFRSF1A, TNFRSF1B, LTBR, TNFRSF4, CD40, FAS, TNFRSF6B, CD27, TNFRSF8, TNFRSF9, TNFRSF10A, TNFRSF10B, TNFRSF10C, TNFRSF10D, TNFRSF11A, TNFRSF11B, TNFRSF12A, TNFRSF13B, TNFRSF13C, TNFRSF14, NGFR, TNFRSF17, TNFRSF18, TNFRSF19, TNFRSF21, and TNFRSF25.

In certain embodiments, transgenic animals comprise one or more genes encoding drug metabolizing enzymes including, but not limited to, CYP1A1, CYP1A2, CYP1B1, CYP2A6, CYP2A13, CYP2A7, CYP2B6, CYP2C8, CYP2C9, CYP2C18, CYP2C19, CYP2D6, CYP2E1, CYP2F1, CYP2J2, CYP2R1, CYP2S1, CYP2U1, CYP2W1, CYP3A4, CYP3A5, CYP3A7, CYP3A43, CYP4A11, CYP4A22, CYP4B1, CYP4F2, CYP4F3, CYP4F8, CYP4F11, CYP4F12, CYP4F22, CYP4V2, CYP4X1, CYP4Z1, CYP5A1, CYP7A1, CYP7B1, CYP8A1, CYP8B1, CYP11A1, CYP11B1, CYP11B2, CYP17A1, CYP19A1, CYP20A1, CYP21A2, CYP24A1, CYP26A1, CYP26B1, CYP26C1, CYP27A1, CYP27B1, CYP27C1, CYP39A1, CYP46A1, CYP51A1, and VKORC1.

In other embodiments, the present invention can also be applied to express one or more chromatin/DNA modifying genes including, but not limited to, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, DNMT1, DNMT3A and, DNMT3B

One or more inflammatory genes can also be used in the context of the present invention including, but not limited to, MAPK14, ICAM1, ITGAL, ITGAM, ITGB1, ITGB2, NFKB1, NOS2, MAPK1, MAPK3, MAPK8, VCAM1, TLR1, TLR3, TLR4, TLR6, TLR7, TLR8, TLR9, IRAK2, MYD88, TRAF6, CD14, CXCR4, CRP, CCL2, NFKB1, CAMP, HMOX1, TREM1, PI3K, PIK3CA, PTEN, AKT1, AKT2, MTOR, JAK 1-3, TYK2, and CCR5.

Accordingly, in one aspect, the present invention provides systems of transgenic non-human animals. In one embodiment, a system comprises (a) a transgenic non-human animal comprising a transgene encoding a wildtype human protein, wherein the protein is biologically active in the animal; and (b) at least one transgenic non-human animal comprising a transgene encoding a variant human protein, wherein the protein is biologically active in the animal and wherein the variant comprises one or more single nucleotide polymorphisms (SNPs). In particular embodiments, the transgenic non-human animal is murine, bovine, ovine, porcine, avian or piscine. In other specific embodiments, the human protein is a cytokine, cytokine receptor, TNF receptor, drug metabolizing enzyme, inflammatory protein or chromatin/DNA modifying protein.

In certain embodiments, the transgene is a bacterial artificial chromosome (BAC) comprising a nucleotide sequence encoding the wildtype or variant human protein. In other embodiments, the transgene encoding the wildtype or variant human protein comprises the non-coding regulatory regions of the human protein. In further embodiments, the one or more SNPs are located in the promoter of the nucleotide sequence encoding the variant human protein.

In a specific embodiment, a system comprises (a) a transgenic mouse comprising a transgene encoding a wildtype human protein, wherein the protein is biologically active in the animal; and (b) at least one transgenic mouse comprising a transgene encoding a variant human protein, wherein the protein is biologically active in the mouse and wherein the variant comprises one or more SNPs. The human protein can be a cytokine, cytokine receptor, TNF receptor, drug metabolizing enzyme, inflammatory gene or chromatin/DNA modifying gene. In a specific embodiment, the human protein is interleukin-10 (IL-10). In another specific embodiment, the transgene is a bacterial artificial chromosome (BAC) comprising a nucleotide sequence encoding the wildtype or variant human protein. In yet another embodiment, the transgene encoding the wildtype or variant human protein comprises the non-coding regulatory regions of the human protein. In certain embodiments, the one or more SNPs are located in the promoter of the nucleotide sequence encoding the variant human protein.

Although the present invention is described in the context of using bacterial artificial chromosomes (BAC), any vector that allows the propagation of large DNA fragments can be used including, but not limited to, yeast artificial chromosomes (YAC) and phage artificial chromosomes (PAC). Indeed, the use of large genomic fragments, cloned as YACs, PACs and BACs can be used to generate the transgenic mice described herein.

In cases where it is unknown if the human gene(s) protein product(s) will functionally interact with endogenous mouse substrate(s), the capacity of the human protein(s) to function in mouse cells can be determined using in vitro experiments. The experimental approach to determine function will vary depending on the substrate. For example, if the human target protein(s) are a cell surface receptor, an appropriate experiment would be to stimulate human cells expressing the target human receptor with the orthologous mouse ligand (recombinant) and measure an expected outcome (i.e., phosphorylation of substrates downstream of the human receptor, induction of target genes, etc.). If it is determined that the human target protein(s) do not functionally interact with mouse substrates, an alternate strategy will employed to assure in vivo function.

More specifically, different approaches may be used to rebuild/replace biologically functional human components of endogenous mouse pathways. In the example used above, the human ligand/substrate for the target human gene(s) will be introduced into mice. This may be done by transgenesis, transduction of viral derived vectors, genome editing (i.e., nuclease-mediated), or by knock-in of the human gene coding regions into the homologous mouse locus. These genetic modifications may be combined at the time of oocyte injection. Oocytes from mouse strains other than C57BL/6 may be used to increase efficiency. One of ordinary skill in the art can assay the functionality of the human protein with the endogenous mouse substrate and, if necessary, rebuild/replace biologically functional human components of endogenous mouse pathway, with undue experimentation.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Example 1

### Construction of the original BAC (hIL10BAC-ATA)

We initiated a database search to identify a human bacterial artificial chromosome (BAC) derived from chromosome 1 in which the IL10 gene is situated approximately at the midpoint. The reason for this is to increase the likelihood that there would be a sufficient amount of genomic DNA surrounding the MO gene to reconstitute normal expression. Data from other BAC transgenic models suggest that a minimum of 50 kb of genomic sequence flanking a given gene is required to recapitulate normal expression. Heintz N. BAC to the future: the use of bac transgenic mice for neuroscience research. Nat Rev Neurosci 2001; 2:861-870. We identified a contig (accession number NT_{—}021877) that consisted of 16,682,800 bp of genomic sequence. Blast searches confirmed the presence of the human genes encoding MAPKAPK2, IL10, and IL19. A BAC clone (RP11-262N9) was identified to contain these genes within the genomic sequence available for contig NT_{—}021877, using NCBI's clone registry searches restricted to human chromosome 1 (http://www.ncbi.nlm.nih.gov/genome/cyto/hbrc.shtml). The location of the BAC was identified in contig NT_{—}021877 by end-sequence information for BAC clone-ID RP11-262N9 based on the accession numbers AQ481134 (SP6), and AQ481138 (T7). Blast searches with end-sequences allowed us to identify the exact size (181 kb) and location of the BAC on chromosome 1 (based on public database nomenclature for numbering chromosome 1: 203,246,000-203,427,000). The BAC clone RP11-262N9 was ordered from BAC PAC Resources (Oakland, Calif.) and was grown from a stab in 1 L of LB broth under chloramphenicol selection, and BAC DNA was isolated by cesium chloride gradient (Lofstrand Labs Limited, Gaithersurg, Md.). The sequence of the hIL10BAC-ATA construct is shown in SEQ ID NO:1. An example of a reference genome for NT_{—}021877 is shown in SEQ ID NO:3.

To release the BAC from the backbone vector (pBACe3.6), the DNA was subjected to NotI digestion. The purified BAC following NotI digestion was 175 kb in length. The BAC DNA was directly injected into fertilized oocytes of C57BL/6 mice. We chose C57BL/6 to eliminate the need to backcross from hybrid strains. See Bygrave A E, Rose K L, Cortes-Hernandez J et al. Spontaneous Autoimmunity in 129 and C57BL/6 Mice-Implications for Autoimmunity Described in Gene-Targeted Mice. PLoS Biology 2004; 2:e243; and McVicar D W, Winkler-Pickett R, Taylor L S et al. Aberrant DAP12 signaling in the 129 strain of mice: implications for the analysis of gene-targeted mice. J Immunol. 2002; 169:1721-1728 Ten of 100 founder mice screened positive for the transgene by Southern blot analysis. Of the 10 founders, 7 survived and were mated with wildtype (WT) C57BL/6 breeders to expand each colony.

### Example 2

### Construction of the New BAC (hIL10BAC-GCC)

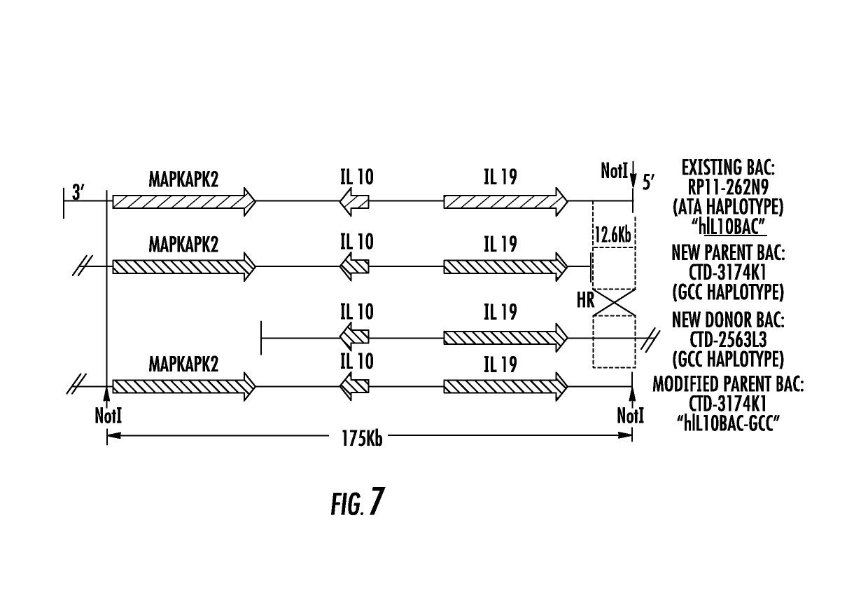

We screened BAC libraries for clones containing the “GCC” haplotype in the IL10 locus and identified a particular clone (CTD-3174K1) that was very close in size to the original hIL10BAC, to create a new “GCC” strain. We generated a construct of the exact length of the original hIL10BAC construct to exclude any potential bias that could be introduced by differences in construct size and DNA content except for the SNPs carried by the different IL10 alleles. To replace the “missing 12.6Kb” of sequence in the “parent BAC” (CTD-3174K1), we identified a “donor BAC” that was from the same library which contained the IL10 promoter. It was important the donor BAC contained the IL10 promoter so that we could verify the GCC allele given that we did not know if the library was derived from an individual heterozygous at this locus. The experimental strategy used to create the GCC strain is illustrated in FIG. 7. In brief, the 12.6Kb fragment from the donor BAC contained the IL10 promoter so that we could verify the GCC allele given that we did not know if the library was derived from an individual heterozygous at this locus. The 12.6Kb fragment from the donor BAC clone (CTD-2563L3) was inserted in frame into the parent BAC by homologous recombination creating a full-length “modified parent BAC” hIL10BAC-GCC construct. The construct was liberated from cloning vector by NotI digestion. Additional genomic sequence 3′ to MAPKAPK2 was removed by an endogenous NotI site (which we confirmed to be free of SNPs). The same endogenous NotI site was used to create the original hIL10BAC construct. The sequence of the hIL10BAC-GCC construct is shown in SEQ ID NO:2.

### Fusion of a 12.6 kb DNA fragment to the 3′ end of BAC CTD-3174K1

Check on BACs.

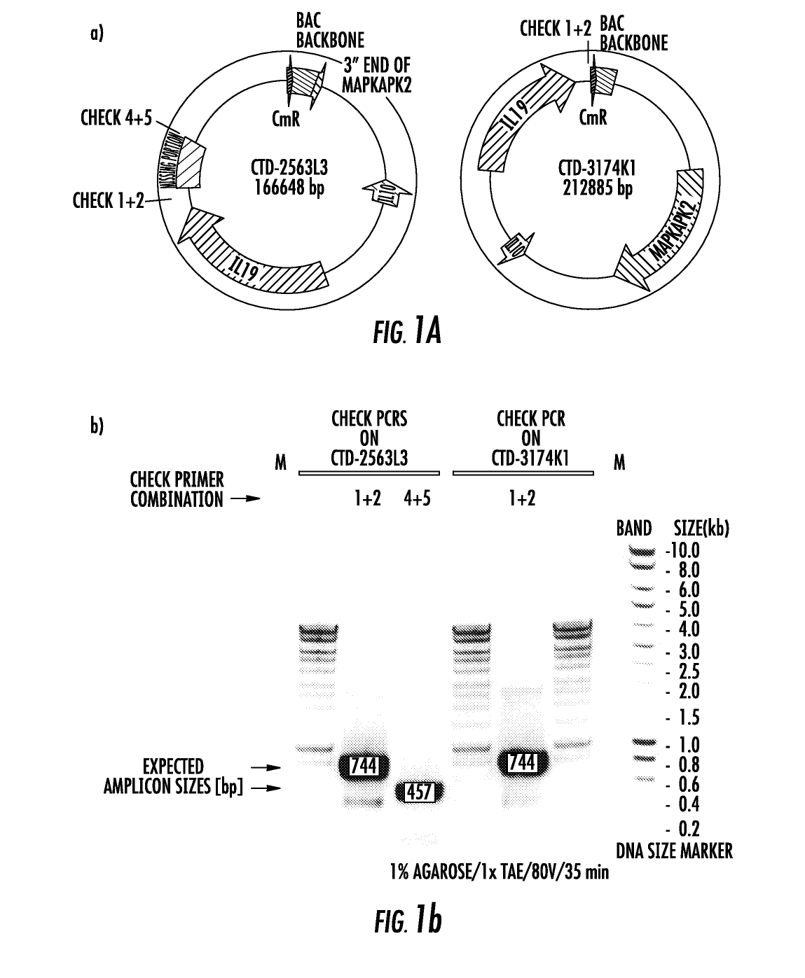

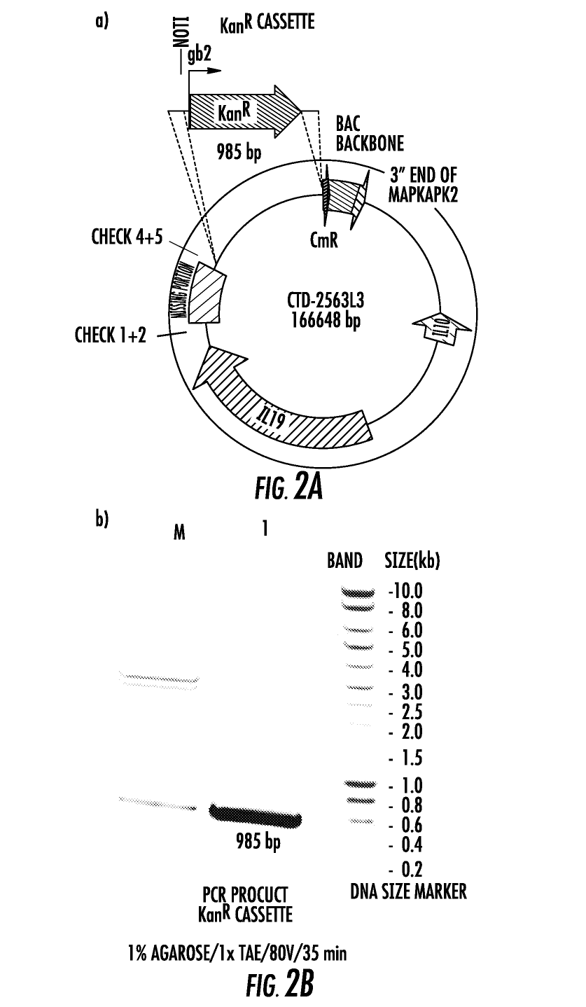

In order to verify integrity, BACs CTD-2563L3 and CTD-3174K1 were subjected to PCR analysis with primers spanning the junctions of the planned modifications. The expected amplification products were obtained for all of the three reactions (FIG. 1).

Generation of the KanR Replacement Cassettes.

In the first stage of the project it was necessary to remove 31 kb of DNA spacing the “missing portion” from the BAC backbone in the donor BAC CTD-2563L3. For that purpose, a replacement cassette containing a Kanamycin resistance gene under control of the prokaryotic gb2 promoter was generated by PCR. This enabled positive selection on recombination events. Homology arms (50 bp) featuring sequence homologies to the regions adjacent to the DNA stretch to be deleted were introduced with the primers used for the cassette synthesis.

Generation of a Truncated CTD-2563L3 BAC Derivative.

The replacement cassette (985 pb) was gel-purified and electroporated into E. coli DH10B harboring BAC CTD-2563L3 and proficient for pRed/ETTet. Red/ET recombination was induced by adding L-Arabinose to the cultures and a temperature shift to 37° C., samples from which L-Arabinose was omitted were used as negative controls. After electroporation, cultures were plated on LB plates containing 15 μg/mL Chloramphenicol and 15 μg/mL Kanamycin. The next day the number of colonies was determined About 170 colonies were obtained. Twelve clones were randomly chosen for further testing. No growth was observed on negative control plates with cultures that were not induced by L-Arabinose.

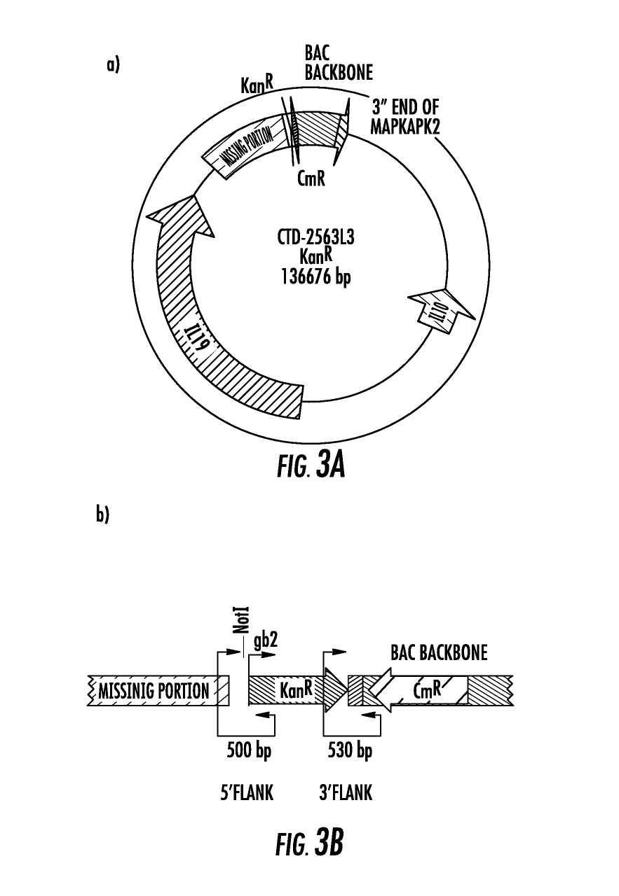

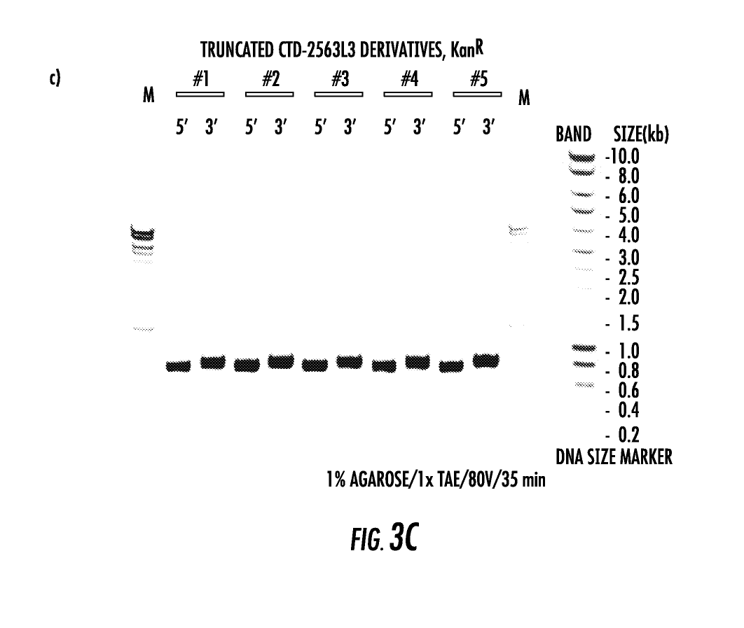

Putative recombinants were subjected to PCR screening across the junctions of the KanR cassette. Correctly recombined clones showed up with a 530 bp and a 500 bp amplificate on the 5′ and the 3′ flank, respectively (FIG. 3). FIG. 3c shows five representative results.

Generation of a Linear Low Copy AmpR-Minimal Vector and Subcloning of the “Missing Portion” from BAC CTD-2563L3-KanR.

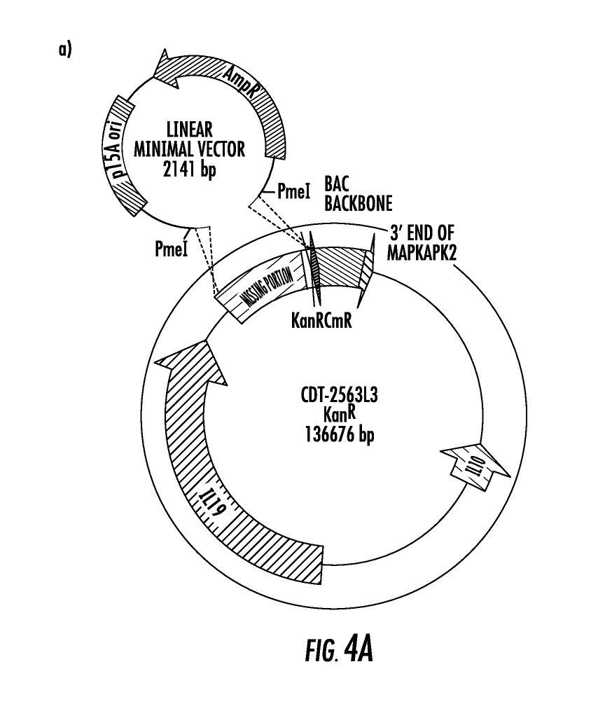

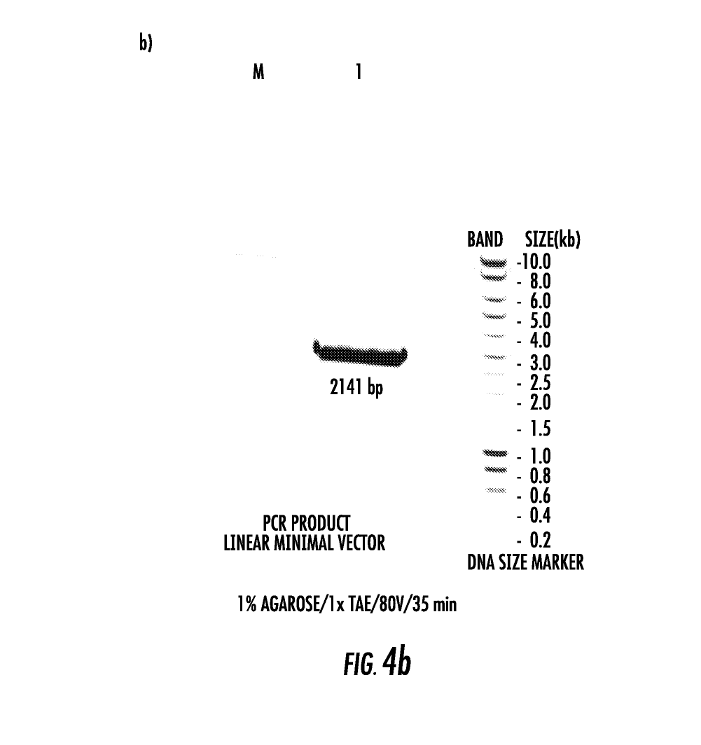

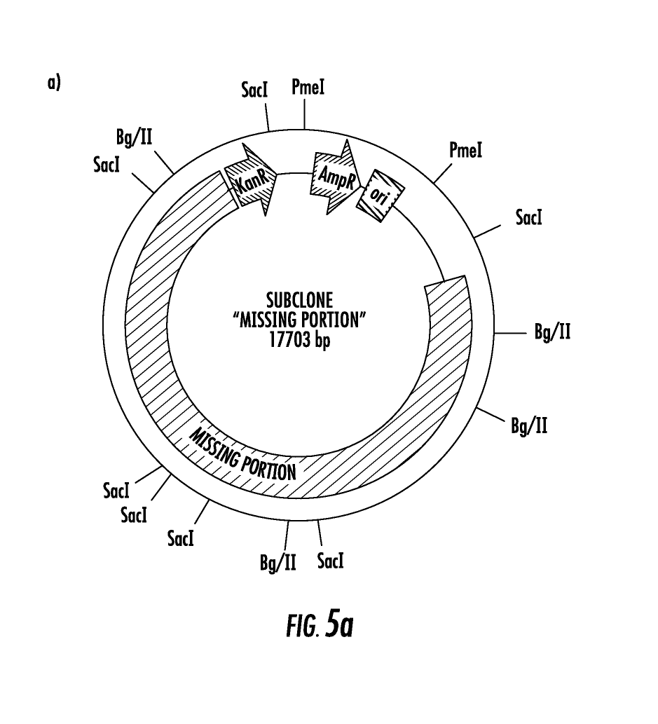

In the next stage of the project the “missing portion” together with the KanR gene was subcloned into a p15A based low copy minimal vector. For that purpose a linear minimal vector (2141 bp) was generated by PCR on pACYC177. Besides two terminal 50 bp homology arms these primers introduced PmeI recognition sites (FIG. 4a).

The minimal vector (2141 pb) was gel-purified and electroporated into E. coli DH10B harboring BAC CTD-2563L3-KanR and proficient for pRed/ETTet. Red/ET recombination was induced by adding L-Arabinose to the cultures and a temperature shift to 37° C., samples from which L-Arabinose was omitted were used as negative controls. After electroporation, cultures were plated on LB plates containing 50 μg/mL Ampicillin and 15 μg/mL Kanamycin. The next day the number of colonies was determined About 860 colonies were obtained. Twelve clones were randomly chosen for the preparation of small scale cultures and glycerol stocks, six of them for immediate further testing. The growth of seven colonies was observed on negative control plates with cultures that were not induced by L-Arabinose.

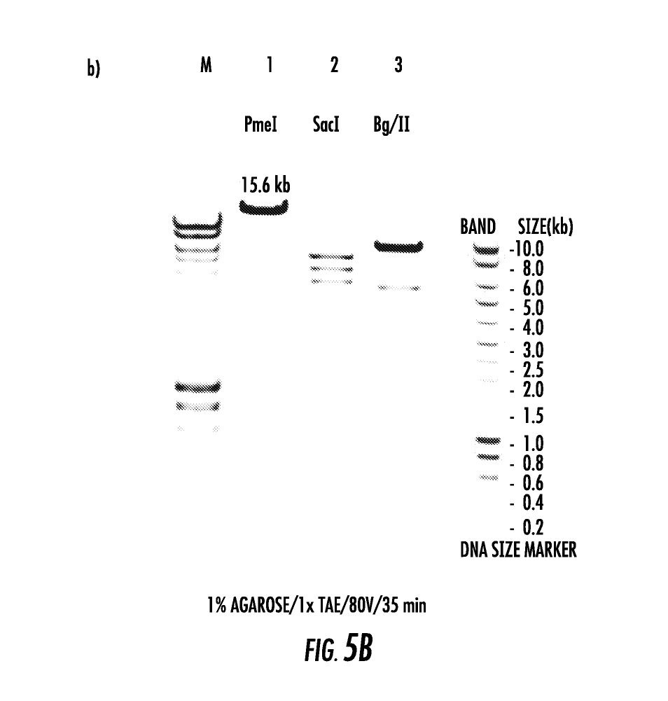

Six subclone candidates were subjected to restriction digestions with PmeI, SacI, and BglII (FIG. 5a). All these clones exhibited the same banding pattern, matching the in silico prediction perfectly. This is exemplified by showing only the restriction pattern of subclone number 1 (FIG. 5b).

The 15.6 kb fragment from FIG. 5b was eluted from the gel and directly used for the final recombination step. This was possible since the terminal sections of this DNA, except for the residual four nucleotides of the former PmeI recognition site, are homologue to the very end of the insert of the acceptor BAC CTD-3174K1, and the adjacent 5′ end of the BAC backbone, respectively.

Generation Integration of the “Missing Portion” Fragment into BAC CTD-3174K1.

The PmeI fragment (15.5 kb) was electroporated into E. coli DH10B harboring BAC CTD-3174K1 and proficient for pRed/ETTet. Red/ET recombination was induced by adding LArabinose to the cultures and a temperature shift to 37° C., samples from which L-Arabinose was omitted were used as negative controls. After electroporation, cultures were plated on LB plates containing 15 μg/mL Chloramphenicol and 15 μg/mL Kanamycin. The next day the number of colonies was determined About 150 colonies were obtained. Six clones were randomly chosen for the preparation of small scale cultures and glycerol stocks, six of them for immediate further testing. The growth of six colonies was observed on negative control plates with cultures that were not induced by L-Arabinose.

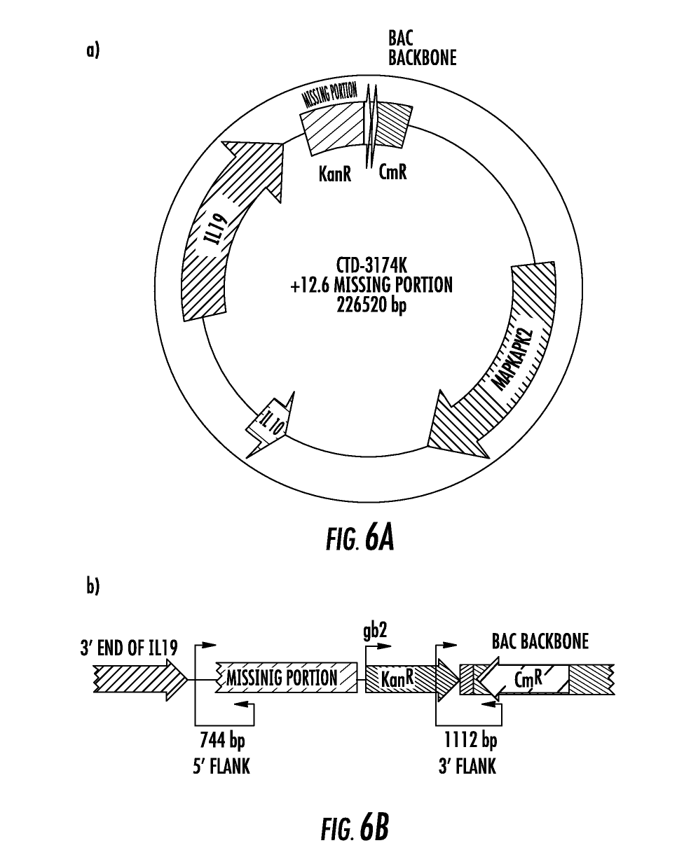

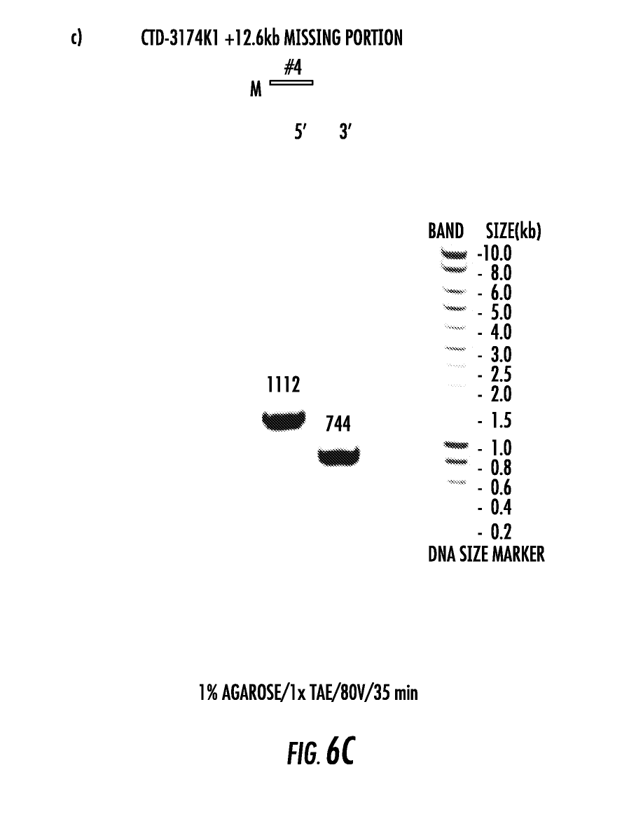

Six of the clones were tested in a flank check PCR (FIG. 6). All clones featured the same results (FIG. 6c shows a result representative for all six clones). Amplificates gained on three of the six tested clones were excised from the gel, eluted and subjected to sequencing reaction. That way the correctness of the integration was proofed (data not shown).

Finally, a pulse field gel electrophoresis was performed with XhoI, FspI, and NotI digestions of the original BAC CTD-3174K1 and the modified BAC CTD-3174K1+12.6 kb missing portion. This side-by-side analysis allowed the comparison of banding pattern of both BACs and with the in silico prediction (data not shown).

As can be seen from FIG. 6, the expected banding pattern was obtained for the two tested BACs. Together with the sequencing results from flank check PCRs it was demonstrated, that die BAC CTD-3174K1+12.6 kb missing portion meets the criteria for a final clone of this project.

In order to make sure the final clone is free of the pRed/ETTet plasmid, single colonies from a loop strike out of clone #4 were tested for their susceptibility to tetracycline. In all cases the tested candidates featured a Tet-sensitive (TetS) phenotype. The glycerol stock delivered to the customer is derived from a single TetS clone.

TABLE 1
hIL10BAC-GCC compared to reference genome for identification
of variants

| Chrom. | | | | |
|---|---|---|---|---|
| No. | POS | ID | REF | ALT |
| 1 | 206854506 | rs4240842 | A | T (replacement of the A |
| | | | | NT by the T NT) |
| 1 | 206854615 | rs4240843 | C | T |
| 1 | 206854886 | rs10625530 | G | GTCA (replacement of the |
| | | | | G NT by GTCA), and so on |
| 1 | 206858172 | . | T | C |
| 1 | 206864573 | rs6683282 | G | A |
| 1 | 206864849 | rs10863777 | T | C |
| 1 | 206864849 | . | T | TATATATATATATATATATAC |
| | | | | (SEQ ID NO: 14) |
| 1 | 206868266 | rs4539133 | A | G |
| 1 | 206868354 | rs4274064 | C | G |
| 1 | 206869116 | rs34268218 | TC | T |
| 1 | 206872487 | rs4845130 | G | A |
| 1 | 206873734 | rs17350838 | C | T |
| 1 | 206874251 | rs6540548 | A | G |
| 1 | 206875176 | rs72220714 | T | TAA |
| 1 | 206875514 | rs4240844 | C | T |
| 1 | 206875697 | rs4240845 | T | C |
| 1 | 206876797 | rs10863787 | C | G |
| 1 | 206876806 | rs10863788 | G | A |
| 1 | 206877337 | rs12060808 | C | T |
| 1 | 206879122 | rs4072677 | G | T |
| 1 | 206879458 | rs10605164 | CTTAT | C |
| 1 | 206884655 | rs6676599 | C | T |
| 1 | 206885839 | rs11119355 | T | G |
| 1 | 206886307 | . | T | TCACACA T TCACACACA |
| | | | | (SEQ ID NO: 15) The |
| | | | | underlined NTs are |
| | | | | added on both sides of |
| | | | | the reference NT “T.” |
| 1 | 206887042 | rs7530164 | T | C |
| 1 | 206888109 | rs34899945 | C | CA |
| 1 | 206889742 | rs4325131 | T | C |
| 1 | 206890435 | rs6669284 | G | A |
| 1 | 206890449 | rs4256810 | T | C |
| 1 | 206891877 | rs71570029 | CAAAAA | C |
| 1 | 206896255 | rs28394820 | T | C |
| 1 | 206896342 | rs4240846 | A | G |
| 1 | 206896622 | rs4240847 | C | A |
| 1 | 206896750 | rs4240848 | T | C |
| 1 | 206896876 | rs4845131 | G | T |
| 1 | 206897481 | rs4607880 | A | G |
| 1 | 206897727 | rs6540574 | G | A |
| 1 | 206897837 | rs4845132 | T | C |
| 1 | 206899052 | rs11119385 | A | G |
| 1 | 206900590 | rs11119389 | T | G |
| 1 | 206901367 | rs34647668 | GT | G |
| 1 | 206901382 | rs11119390 | C | G |
| 1 | 206904464 | rs10863805 | C | T |
| 1 | 206906625 | rs10610415 | ATGTG | A ATGTG ATG |
| | | | | The underlined NTs are |
| | | | | added on both sides of |
| | | | | the reference NT “ATGTG.” |
| 1 | 206906943 | . | T | C |
| 1 | 206912772 | rs144807922 | G | GTCC |
| 1 | 206913780 | rs3935632 | G | A |
| 1 | 206914016 | rs10863812 | T | C |
| 1 | 206915418 | rs4844552 | A | G |
| 1 | 206916362 | rs6540582 | C | T |
| 1 | 206917285 | rs72314394 | TTCTCTTTCTTTC | T TTCTCTTTCTTTCCTTCCT TTCCT |
| | | | CTTCCT | (SEQ ID NO: 17) |
| | | | (SEQ ID NO: 16) | |
| 1 | 206917326 | rs12138730 | C | T |
| 1 | 206917334 | rs71515110 | C | T |
| 1 | 206917338 | rs6668374 | C | T |
| 1 | 206917342 | rs12138733 | C | T |
| 1 | 206917400 | rs72371918 | CTCCTTCCTTCCT | C |
| | | | TCCTTCCT | |
| | | | (SEQ ID NO: 18) | |
| 1 | 206917421 | . | TC | T |
| 1 | 206917480 | rs61610737 | C | CT |
| 1 | 206918383 | rs4483409 | A | G |
| 1 | 206918855 | rs111264125 | T | TA |
| 1 | 206920546 | rs6540586 | A | G |
| 1 | 206920804 | . | CTA | C |
| 1 | 206920820 | rs67618268 | A | T |
| 1 | 206920822 | rs79580208 | A | T |
| 1 | 206923478 | rs11119447 | T | C |
| 1 | 206924226 | rs6540587 | C | T |
| 1 | 206924244 | rs72755097 | G | C |
| 1 | 206924289 | rs7536492 | G | A |
| 1 | 206924536 | rs11119449 | T | A |
| 1 | 206924662 | rs71846636 | A | AAC |
| 1 | 206925681 | rs11119451 | G | C |
| 1 | 206925844 | rs7556267 | T | C |
| 1 | 206925881 | rs13373997 | A | T |
| 1 | 206925893 | rs66488234 | C | T |
| 1 | 206925934 | rs67575336 | T | C |
| 1 | 206925977 | rs57859555 | T | C |
| 1 | 206926004 | rs146809685 | TTTCC | T |
| 1 | 206926846 | rs6658896 | T | C |
| 1 | 206927288 | . | C | CTT |
| 1 | 206927516 | rs6692511 | T | C |
| 1 | 206929182 | rs7418268 | T | G |
| 1 | 206930055 | rs6699203 | A | C |
| 1 | 206932418 | rs186924884 | T | C |
| 1 | 206932451 | rs4390174 | A | G |
| 1 | 206935483 | rs114952863 | G | A |
| 1 | 206936197 | rs138082438 | C | CTCCTTTCCTTTCCTTTCCTTTCCT |
| | | | | T C CCTT (SEQ ID NO: 19) |
| 1 | 206936262 | rs149127784 | C | T |
| 1 | 206936266 | rs5028660 | C | T |
| 1 | 206936266 | rs138438672 | C | CCTTTCTTT C CCTTCCTTTCTTT |
| | | | | (SEQ ID NO: 20) |
| 1 | 206936345 | rs72420444 | CTCTTTCTT | C |
| 1 | 206936432 | rs71152492 | C | CT |
| 1 | 206939904 | rs3024505 | G | A |
| 1 | 206940310 | rs3024502 | C | T |
| 1 | 206940766 | rs193120806 | G | C |
| 1 | 206940831 | rs3024500 | A | G |
| 1 | 206941864 | rs3024496 | A | G |
| 1 | 206942413 | rs3024495 | C | T |
| 1 | 206943351 | rs3024494 | T | C |
| 1 | 206943713 | rs1878672 | G | C |
| 1 | 206943968 | rs3024493 | C | A |
| 1 | 206944233 | rs1554286 | A | G |
| 1 | 206944645 | rs1518111 | T | C |
| 1 | 206944861 | rs1518110 | A | C |
| 1 | 206945046 | rs3024491 | C | A |
| 1 | 206945311 | rs3024490 | A | C |
| 1 | 206945381 | rs2222202 | G | A |
| 1 | 206946407 | rs1800872 | T | G |
| 1 | 206946634 | rs1800871 | A | G |
| 1 | 206946897 | rs1800896 | T | C |
| 1 | 206946931 | rs3980910 | T | TTGTGTGTGTGTG |
| | | | | (SEQ ID NO: 21) |
| 1 | 206947167 | rs1800893 | C | T |
| 1 | 206948553 | rs6693899 | G | T |
| 1 | 206949365 | rs1800890 | A | T |
| 1 | 206950002 | rs3001101 | A | C |
| 1 | 206950003 | rs2945417 | T | C |
| 1 | 206950608 | rs11119514 T | C | |
| 1 | 206950621 | rs11119515 T | G | |
| 1 | 206952204 | rs10494879 C | G | |
| 1 | 206952748 | rs6676671 | T | A |
| 1 | 206954566 | rs12123181 G | A | |
| 1 | 206954759 | rs57060549 C | CA | |
| 1 | 206954784 | rs144436741G | A | |
| 1 | 206954794 | . | G | A |
| 1 | 206955315 | . | TGTTATATATACA | TAGTTATATATACATATATATGTA |
| | | | TATATATGTATAT | TATGTTATATATACATATATATGT |
| | | | GTTATATATACAT | ATATAGTTATATATACATATATAT |
| | | | ATATATGTATATA | GTATAT |
| | | | GTTATATATACAT | TGTTATATATACATATATATGTAT |
| | | | ATATATGTATAT | ATGTTATATATACATATATATGTA |
| | | | (SEQ ID NO: 22) | TATAGTTATATATACATATATATG |
| | | | | TATAT T (SEQ ID NO: 23) |
| 1 | 206955340 | rs11273390 | T | TA |
| 1 | 206955408 | rs145665559 | ATG | A |
| 1 | 206955651 | rs6686931 | T | C |
| 1 | 206957092 | rs6667202 | C | A |
| 1 | 206957449 | rs4072226 | T | C |
| 1 | 206957558 | rs4072227 | C | T |
| 1 | 206965833 | rs147597174 | G | A |
| 1 | 206970628 | rs11280588 | G | GTAGAATTC |
| 1 | 206980503 | rs186302513 | C | T |
| 1 | 206988295 | rs144443711 | C | A |
| 1 | 206988346 | rs147404592 | G | C |
| 1 | 206988433 | rs1400988 | T | C |
| 1 | 206991253 | rs4440852 | C | A |
| 1 | 206991401 | rs2883034 | G | A |
| 1 | 206992162 | rs11373916 | C | CT |
| 1 | 206992801 | rs11119629 | T | C |
| 1 | 206992925 | rs113703997 | T | TA |
| 1 | 206994404 | rs2138992 | A | C |
| 1 | 206995478 | rs6685379 | G | A |
| 1 | 206996355 | rs12096695 | G | A |
| 1 | 206997892 | rs2056226 | G | C |
| 1 | 206998706 | rs2056225 | A | T |
| 1 | 206999963 | rs908703 | G | C |
| 1 | 207000060 | rs908704 | G | A |
| 1 | 207000952 | rs4240849 | A | G |
| 1 | 207001000 | rs2883035 | C | T |
| 1 | 207001040 | rs2352794 | C | A |
| 1 | 207001053 | rs147886503 | AAC | A |
| 1 | 207001190 | rs6540645 | G | C |
| 1 | 207001709 | rs1028181 | T | C |
| 1 | 207001879 | rs1028182 | T | C |
| 1 | 207002356 | rs5780365 | C | CT |
| 1 | 207003319 | rs4845143 | G | T |
| 1 | 207003374 | rs12022129 | G | A |
| 1 | 207003553 | rs6660520 | A | G |
| 1 | 207003577 | rs6663563 | T | C |
| 1 | 207003583 | rs6660537 | A | G |
| 1 | 207003919 | rs113193509 | G | GTT |
| 1 | 207003956 | rs7513988 | C | T |
| 1 | 207004069 | rs7518426 | A | G |
| 1 | 207005321 | rs11119670 | C | T |
| 1 | 207006214 | rs2243156 | C | G |
| 1 | 207007641 | rs2243158 | C | G |
| 1 | 207009388 | rs2243168 | T | A |
| 1 | 207009910 | rs2243170 | T | C |
| 1 | 207010626 | rs2073186 | T | C |
| 1 | 207010834 | rs2243171 | A | G |
| 1 | 207010984 | rs2243172 | C | CA |
| 1 | 207011485 | rs2243174 | G | A |
| 1 | 207012081 | . | TTTCCC | T |
| 1 | 207014472 | rs2243188 | A | C |
| 1 | 207014776 | rs960326 | C | T |
| 1 | 207015957 | rs2243191 | T | C |
| 1 | 207016225 | rs2243193 | A | G |
| 1 | 207016648 | rs4845144 | G | A |
| 1 | 207017805 | rs149441664 | GTAATA | G |
| 1 | 207018394 | rs7532642 | T | C |
| 1 | 207018432 | rs112204403C | CA | |
| 1 | 207021819 | rs200352185 | CATACTGAAGA | C |
| | | | (SEQ ID NO: 24) | |
| 1 | 207021858 | rs71152496 | G | GTTT G GTTTT |
| | | | | (SEQ ID NO: 25) |
| 1 | 207022139 | rs4313398 | A | G |
| 1 | 207024138 | rs76757827 | G | A |
| 1 | 207026356 | rs143405835 | G | C |
| 1 | 207026382 | rs148368340 | C | T |
| 1 | 207026835 | rs1713233 | T | C |
| 1 | 207027478 | rs34492629 | TA | T |
| 1 | 207028395 | rs34653076 | CTT | C CTT CT |
| 1 | 207030985 | rs1033272 | T | C |
| 1 | 207031187 | . | G | A |
| 1 | 207031737 | rs34778683 | CA | C |
| 1 | 207032089 | rs11119726 | T | A |
| 1 | 207032268 | rs570249 | G | A |
| 1 | 207032839 | rs552760 | C | G |
| 1 | 207033330 | rs150239537 | T | C |
| 1 | 207033714 | rs523435 | A | G |
| 1 | 207033778 | rs118019112 | G | A |
| 1 | 207033965 | . | T | TAAAA T |
| | | | | TATATATATATATATATATATATA |
| | | | | TATATAAAA (SEQ ID NO: 26) |
| 1 | 207034352 | rs11808756 | T | C |

TABLE 2
hIL10BAC-ATA compared to reference genome for identification
of variants

| Chrom. | | | | |
|---|---|---|---|---|
| No. | POS | ID | REF | ALT |
| 1 | 206850230 | rs7522247 | A | G |
| 1 | 206858172 | . | T | C |
| 1 | 206888109 | rs34899945 | C | CA |
| 1 | 206954784 | rs144436741 | G | A |
| 1 | 206964646 | rs71877178 | G | GT |
| 1 | 207026835 | rs1713233 | T | C |
| 1 | 207027478 | rs34492629 | T | TA |
| 1 | 207028395 | rs34653076 | CT | C |
| 1 | 207030985 | rs1033272 | T | C |
| 1 | 207031737 | rs34778683 | CA | C |
| 1 | 207032268 | rs570249 | G | A |
| 1 | 207032751 | rs574773 | C | T |
| 1 | 207032839 | rs552760 | C | G |
| 1 | 207033714 | rs523435 | A | G |
| 1 | 207033951 | rs71570041 | C | CATATATATATATATATATAT C |
| | | | | CATATATATATATATAT |
| | | | | (SEQ ID NO: 27) |

### Example 3

### Construction of Transgenic Mouse System Expressing a Cytokine Receptor

BAC clones containing the genes encoding the human IL23 receptor (IL23R) (human IL23R and IL12RB) genes and regulatory regions is identified and used to created hIL23RBAC transgenic mice. The DNA is subjected to restriction enzyme digestion and the insert is purified for microinjection. The purified BACs are of appropriate length and are injected into fertilized mice (e.g., C57BL/6). The founder mice are screened for the transgenes by Southern blot analysis. The surviving founder mice are mated with wildtype (WT) C57BL/6 breeders to expand each colony. hIL23RBAC transgenic mice are created for wildtype hIL23R components as well as one or more SNP haplotypes within hIL23R. In certain embodiments, the inserts for the wildtype and SNP versions of hIL23R are the same length.

Mice from separate litters in each founder line are used to assess transgene copy numbers. Quantitative PCR assays are designed to detect the hIL23RBAC transgene. Data are analyzed and compared to a standard curve of known genes with varying copy numbers.

Basal tissue expression of human and endogenous mouse IL23R in hIL23R mice is examined. It is expected that the human IL23R BAC cassette supports appropriate human IL23R expression. It is also expected that hIL23R regulates target genes in vivo. To assure a fully functional hIL23R, human IL-23 (p19 and p40) is introduced by gene targeting to replace the endogenous mouse genes. Animals are backcrossed to generate genetically humanized mice with a fully functional IL-23/IL23R signaling pathway.

### Example 4

### Construction of Transgenic Mouse System Expressing a TNF Receptor

In one embodiment, transgenic mice that express functional human wildtype and variant FAS are constructed. The enzyme is encoded by the TNFRSF6 gene. A BAC clone containing the FAS gene and regulatory regions is identified and used to created hFASBAC transgenic mice. The DNA is subjected to restriction enzyme digestion and the insert is purified for microinjection. The purified BAC is of appropriate length and is injected into fertilized mice (e.g., C57BL/6). The founder mice are screened for the transgene by Southern blot analysis. The surviving founder mice are mated with wildtype (WT) C57BL/6 breeders to expand each colony. hFASBAC transgenic mice are created for wildtype hFAS as well as one or more SNPs within hFAS. In certain embodiments, the inserts for the wildtype and SNP versions of hFAS are the same length.

Mice from separate litters in each founder line are used to assess transgene copy numbers. Quantitative PCR assays are designed to detect the hFASBAC transgene. Data are analyzed and compared to a standard curve of known genes with varying copy numbers.

Basal tissue expression of human and endogenous mouse FAS in hFAS mice is examined. It is expected that the human FAS BAC cassette supports appropriate human FAS expression. It is also expected that FAS regulates target genes in vivo.

### Example 5

### Construction of Transgenic Mouse System Expressing a Drug Metabolizing Enzyme

In one embodiment, transgenic mice that express functional human wildtype and variant vitamin K epoxide reductase complex subunit 1 are constructed. The enzyme is encoded by the VKORC1 gene. A BAC clone containing the VKORC1 gene and regulatory regions is identified and used to created hVKORC1BAC transgenic mice. The DNA is subjected to restriction enzyme digestion and the insert is purified for microinjection. The purified BAC is of appropriate length and is injected into fertilized mice (e.g., C57BL/6). The founder mice are screened for the transgene by Southern blot analysis. The surviving founder mice are mated with wildtype (WT) C57BL/6 breeders to expand each colony. hVKORC1BAC transgenic mice are created for wildtype hVKORC1 as well as one or more SNPs within hVKORC1. In certain embodiments, the inserts for the wildtype and SNP versions of hVKORC1 are the same length.

Mice from separate litters in each founder line are used to assess transgene copy numbers. Quantitative PCR assays are designed to detect the hVKORC1BAC transgene. Data are analyzed and compared to a standard curve of known genes with varying copy numbers.

Basal tissue expression of human and endogenous mouse VKORC1 in hVKORC1 mice is examined. It is expected that the human VKORC1 BAC cassette supports appropriate human VKORC1 expression. It is also expected that VKORC1 regulates target genes in vivo.

### Example 6

### Construction of Transgenic Mouse System Expressing a Drug Metabolizing Enzyme

In one embodiment, transgenic mice that express functional human wildtype and variant cytochrome P450 2C9 (CYP2C9) are constructed. The enzyme is encoded by the CYP2C9 gene. A BAC clone containing the CYP2C9 gene and regulatory regions is identified and used to created hCYP2C9BAC transgenic mice. The DNA is subjected to restriction enzyme digestion and the insert is purified for microinjection. The purified BAC is of appropriate length and is injected into fertilized mice (e.g., C57BL/6). The founder mice are screened for the transgene by Southern blot analysis. The surviving founder mice are mated with wildtype (WT) C57BL/6 breeders to expand each colony. hCYP2C9BAC transgenic mice are created for wildtype hCYP2C9 as well as one or more SNPs within hCYP2C9. In certain embodiments, the inserts for the wildtype and SNP versions of hCYP2C9 are the same length.

Mice from separate litters in each founder line are used to assess transgene copy numbers. Quantitative PCR assays are designed to detect the hCYP2C9BAC transgene. Data are analyzed and compared to a standard curve of known genes with varying copy numbers.

Basal tissue expression of human and endogenous mouse CYP2C9 in hCYP2C9 mice is examined. It is expected that the human CYP2C9 BAC cassette supports appropriate human CYP2C9 expression. It is also expected that CYP2C9 regulates target genes in vivo.

### Example 7

### Construction of Transgenic Mouse System Expressing an Inflammatory Gene

In one embodiment, transgenic mice that express functional human wildtype and variant Toll-like receptor 4 (TLR4) are constructed. The enzyme is encoded by the TLR4 gene. A BAC clone containing the TLR4 gene and regulatory regions is identified and used to created hTLR4BAC transgenic mice. The DNA is subjected to restriction enzyme digestion and the insert is purified for microinjection. The purified BAC is of appropriate length and is injected into fertilized mice (e.g., C57BL/6). The founder mice are screened for the transgene by Southern blot analysis. The surviving founder mice are mated with wildtype (WT) C57BL/6 breeders to expand each colony. hTLR4BAC transgenic mice are created for wildtype hTLR4 as well as one or more SNPs within hTLR4. In certain embodiments, the inserts for the wildtype and SNP versions of hTLR4 are the same length.

Mice from separate litters in each founder line are used to assess transgene copy numbers. Quantitative PCR assays are designed to detect the hTLR4BAC transgene. Data are analyzed and compared to a standard curve of known genes with varying copy numbers.

Basal tissue expression of human and endogenous mouse TLR4 in hTLR4 mice is examined. It is expected that the human TLR4 BAC cassette supports appropriate human TLR4 expression. It is also expected that TLR4 regulates target genes in vivo.

### Example 8

### Construction of Transgenic Mouse System Expressing a Chromatin/DNA Modifying Gene

In one embodiment, transgenic mice that express functional human wildtype and variant histone deacetylase 2 (HDAC2) are constructed. The enzyme is encoded by the HDAC2 gene. A BAC clone containing the HDAC2 gene and regulatory regions is identified and used to created hHDAC2BAC transgenic mice. The DNA is subjected to restriction enzyme digestion and the insert is purified for microinjection. The purified BAC is of appropriate length and is injected into fertilized mice (e.g., C57BL/6). The founder mice are screened for the transgene by Southern blot analysis. The surviving founder mice are mated with wildtype (WT) C57BL/6 breeders to expand each colony. hHDAC2BAC transgenic mice are created for wildtype hHDAC2 as well as one or more SNPs within hHDAC2. In certain embodiments, the inserts for the wildtype and SNP versions of hHDAC2 are the same length.

Mice from separate litters in each founder line are used to assess transgene copy numbers. Quantitative PCR assays are designed to detect the hHDAC2BAC transgene. Data are analyzed and compared to a standard curve of known genes with varying copy numbers.

Basal tissue expression of human and endogenous mouse HDAC2 in hHDAC2 mice is examined. It is expected that the human HDAC2 BAC cassette supports appropriate human HDAC2 expression. It is also expected that HDAC2 regulates target genes in vivo.

1. A system comprising:
a. a transgenic non-human animal comprising a transgene encoding a wildtype human protein, wherein the protein is biologically active in the animal; and
b. at least one transgenic non-human animal comprising a transgene encoding a variant human protein, wherein the protein is biologically active in the animal and wherein the variant comprises one or more single nucleotide polymorphisms (SNPs).

2. The system of claim 1, wherein the transgenic non-human animal is murine, bovine, ovine, porcine, avian or piscine.

3. The system of claim 1, wherein the human protein is a cytokine, cytokine receptor, TNF receptor, drug metabolizing enzyme, inflammatory protein or chromatin/DNA modifying protein.

4. The system of claim 1, wherein the transgene is a bacterial artificial chromosome (BAC) comprising a nucleotide sequence encoding the wildtype or variant human protein.

5. The system of claim 1, wherein the transgene encoding the wildtype or variant human protein comprises the non-coding regulatory regions of the human protein.

6. The system of claim 1, wherein the one or more SNPs are located in the promoter of the nucleotide sequence encoding the variant human protein.

7. A system comprising:
a. a transgenic mouse comprising a transgene encoding a wildtype human protein, wherein the protein is biologically active in the animal; and
b. at least one transgenic mouse comprising a transgene encoding a variant human protein, wherein the protein is biologically active in the mouse and wherein the variant comprises one or more SNPs.

8. The method of claim 7, wherein the human protein is a cytokine, cytokine receptor, TNF receptor, drug metabolizing enzyme, inflammatory protein or chromatin/DNA modifying protein.

9. The method of claim 7, wherein the human protein is interleukin-10 (IL-10).

10. The system of claim 7, wherein the transgene is a bacterial artificial chromosome (BAC) comprising a nucleotide sequence encoding the wildtype or variant human protein.

11. The system of claim 7, wherein the transgene encoding the wildtype or variant human protein comprises the non-coding regulatory regions of the human protein.

12. The system of claim 7, wherein the one or more SNPs are located in the promoter of the nucleotide sequence encoding the variant human protein.

13. A transgenic non-human animal comprising a transgene encoding a variant human interleukin-10 (IL-10), wherein human IL-10 is biologically active in the animal and wherein the variant comprises one or more SNPs.

14. The transgenic non-human animal of claim 13, wherein the animal is murine, bovine, ovine, porcine, avian or piscine.

15. The transgenic non-human animal of claim 13, wherein the transgene is a bacterial artificial chromosome (BAC) comprising a nucleotide sequence encoding a variant human IL-10.

16. The transgenic non-human animal of claim 13, wherein the one or more SNPs are located in the IL-10 promoter.

17. The transgenic non-human animal of claim 16, wherein the SNP is one or more of rs12569132, rs11809840, rs11809303, rs11808255, rs11802425, rs11802412, rs11799787, rs11585685, rs11579735, rs11119451, rs11119449, rs10494878, rs7556267, rs7548373, rs7537619, rs7519318, rs7512090, rs7418268, rs6692511, rs6682675, rs6673928, rs6668464, rs6668374, rs6540587, rs6540586, rs6540582, rs4844553, rs4844552, rs4579758, rs4483409, and rs11807715.

18. The transgenic non-human animal of claim 13, wherein the one or more SNPs are located in the IL-10 gene.

19. The transgenic non-human animal of claim 18, wherein the SNP is one or more of rs3024496, rs3024491, rs3024490, rs3021094, rs2222202, rs1800896, rs1800894, rs1800893, rs1800891, rs1800872, rs1800871, rs1554286, rs1518111, and rs1518110.

20. The transgenic non-human animal of claim 3, wherein the BAC further comprises a nucleotide sequence encoding mitogen activated protein kinase-activated protein kinase 2 (MK2).

21. The transgenic non-human animal of claim 3, wherein the BAC further comprises a nucleotide sequence encoding IL-19.

22. The transgenic non-human animal of claim 3, wherein the BAC further comprises a nucleotide sequence encoding IL-20.

23. A method for screening a candidate agent for the ability modulate IL-10 expression in the transgenic animal of claim 12 comprising the steps of:
a. administering to a first transgenic animal of claim 1 a candidate agent; and
b. comparing IL-10 expression in the first transgenic animal to IL-10 expression of a second transgenic animal of claim 1 not administered the candidate agent;
wherein a difference in the IL-10 expression in the first transgenic animal administered the candidate agent compared to the second transgenic animal not administered the candidate agent is indicative of a candidate agent that modulates IL-10 expression.

24. The method of claim 23, wherein the transgenic animal further comprises a disease model that is associated with IL-10.

25. The method of claim 24, wherein a difference in disease severity is also compared.

26. A system comprising (a) hIL10BAC-ATA mice and (b) hIL10BAC-GCC mice.

27. A transgenic non-human animal comprising a transgene encoding a human protein known to influence disease susceptibility, wherein the protein is biologically active in the animal.
